(19) United States
(12) Patent Application Publication
(10) Pub. No.: US 2006/0184126 A1
(43) Pub. Date: Aug. 17, 2006

(54) Method and apparatus for controlling fluid flow

(76) Inventor: Karthikanallil Antony, Chesterfield, MO (US)

Correspondence Address: PATRICK W. RASCHE;ARMSTRONG TEASDALE LLP, ONE METROPOLITAN SQUARE, SUITE 2600, ST. LOUIS, MO, 63102-2740, US

(21) Appl. No.: 11/322,729

(22) Filed: Dec. 30, 2005

Related U.S. Application Data
(60) Provisional application No. 60/593,790, filed on Feb. 14, 2005.

(51) Int. Cl. A61M 5/178

(52) U.S. Cl. 604/167.01

(57) ABSTRACT

A fluid flow control device includes a structural body forming a channel. A sheath is coupled to a first end of the structural body and has a tip. The sheath defines a passage in flow communication with the channel. A needle is movably positioned within the channel. The needle is movable between a retracted position seated within the channel and an extended position extending through the passage and outwardly with respect to the tip. A port defines a second passage at an opposing second end of the structural body. The second passage is in flow communication with the channel.

### CROSS REFERENCE TO RELATED APPLICATIONS

This application claims the benefit of U.S. Provisional Application No. 60/593,790, filed Feb. 14, 2005, which is hereby incorporated by reference in its entirety.

### BACKGROUND OF THE INVENTION

This invention relates generally to a method and apparatus for providing fluid flow and, more particularly, to a method and apparatus for transferring fluids, such as blood, between an external fluid source and a patient's body.

During medical and/or clinical procedures, it is often required to draw fluid from a patient's body and/or introduce fluid into the patient's body. For example, procedures, such as dialysis procedures, may require the use of a catheter device for access to the patient's blood flow. The catheter device includes a needle that is a repeatedly, subcutaneously introduced into the patient's body. The repeated introduction of the needle and/or long-term exposure of the needle to the patient's skin and internal tissues are damaging to the patient's skin, surrounding tissue and/or veins, causing pain, irritation and/or excessive bruising.

### BRIEF DESCRIPTION OF THE INVENTION

In one aspect, the present invention provides a fluid flow control device that includes a structural body forming a channel. A sheath is coupled to a first end of the structural body and has a tip. The sheath defines a passage in flow communication with the channel. A needle is movably positioned within the channel. The needle is movable between a retracted position seated within the channel and an extended position extending through the passage and outwardly with respect to the tip. A port defines a second passage at an opposing second end of the structural body. The second passage is in flow communication with the channel.

In another aspect, the present invention provides a fluid flow control system for transferring blood between an external blood source and a patient's systemic circulatory system. The fluid flow control system includes an arteriovenous shunt that defines a blood flow passage. The arteriovenous shunt provides flow communication between an artery and a vein of the systemic circulatory system. The fluid flow control system includes a fluid flow control device including a structural body forming a channel. A sheath is coupled to the structural body and defines a passage in flow communication with the channel. A needle is movably positioned within the channel. The needle is movable between a retracted position seated within the channel and an extended position extending beyond a tip of the sheath. Each of the needle and the sheath is at least partially positioned within the blood flow passage when the needle is in the extended position. The sheath is at least partially positioned within the blood flow passage when the needle is in the retracted position. A fluid transfer tube provides flow communication between the external blood source and the fluid flow control device.

In another aspect, the present invention provides a method for transferring a fluid between an external fluid source and a patient's body. The method includes inserting a needle and a sheath surrounding the needle into a shunt. The sheath is coupled to a fluid flow device structural body that defines a channel. The needle is retracted to remove the needle from within the shunt. The sheath provides continued flow communication between a fluid flow passage formed by the shunt and the channel.

### BRIEF DESCRIPTION OF THE DRAWINGS

FIG. 1 is a top plan view of a fluid flow control system having a needle in an extended position with respect to a sheath;

FIG. 2 is a side view of the fluid flow control system of FIG. 1 with the needle and the sheath inserted into a shunt;

FIG. 3 is a top plan view of the fluid flow control system of FIG. 1 with the needle in a retracted position with respect to the sheath;

FIG. 4 is a side view of the fluid flow control system of FIG. 1 with the needle in a retracted position and the sheath inserted into the shunt;

FIG. 5 is a perspective view of the fluid flow control system of FIG. 1 with the needle in an extended position with respect to the sheath;

FIG. 6 is a perspective view of the fluid flow control system of FIG. 1 with the needle in a retracted position with respect to the sheath;

FIG. 7 is a partial sectional view of a fluid flow control system with a plate engaged with an end wall of a structural body;

FIG. 8 is a partial sectional view of the fluid flow control system of FIG. 7 with the plate disengaged from the end wall;

FIG. 9 is a partial sectional view of the fluid flow control system of FIG. 7 with the plate disengaged from the end wall; and

FIG. 10 is a partial sectional view of the fluid flow control system of FIG. 7 with the plate disengaged from the end wall.

### DETAILED DESCRIPTION OF THE INVENTION

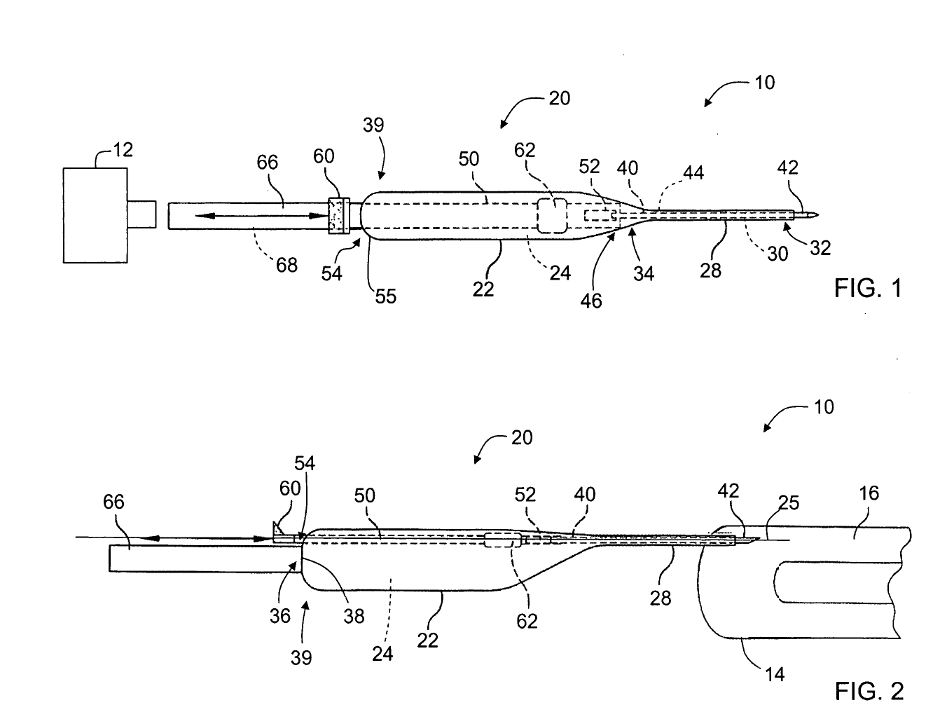

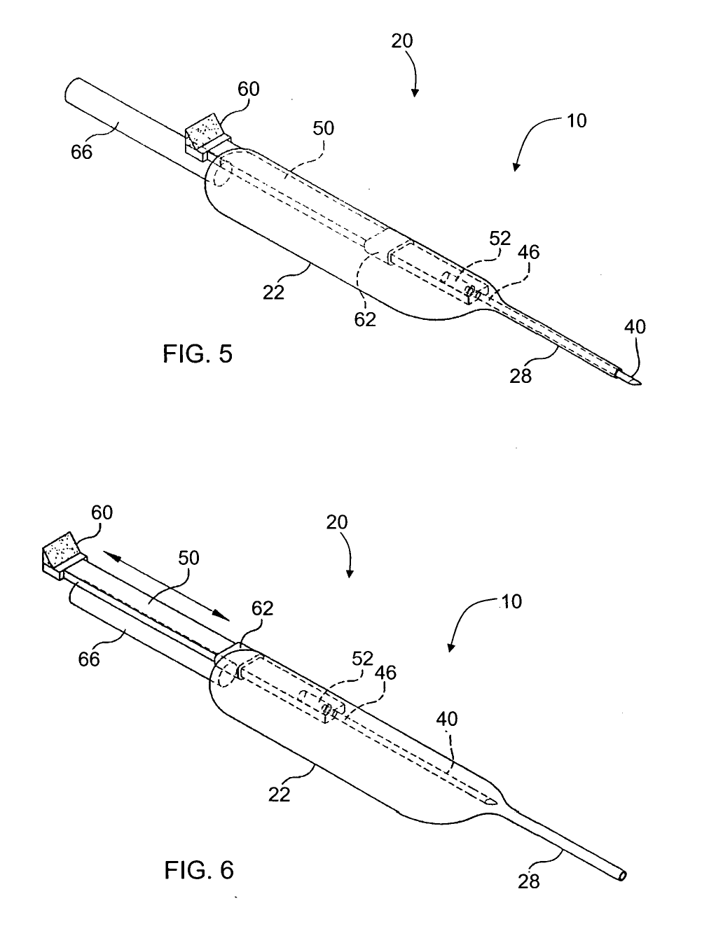

Referring to FIGS. 1-10, the present invention provides a method and apparatus for controlling fluid flow. In one embodiment, a fluid flow control device and a fluid flow control system for transferring fluid, such as blood, between an external device and a patient's body is provided. The fluid flow control device and system of the present invention can be utilized to draw fluids from the patient's body and/or introduce fluids into the patient's body. The present invention is described below in reference to its application in connection with and operation of a fluid flow control system for transferring blood between an external blood source and a patient's body, such as during a dialysis procedure. However, it will be obvious to those skilled in the art and guided by the teachings herein provided that the invention is likewise applicable to any suitable surgical and/or clinical procedure for drawing, collecting, introducing and/or circulating a fluid, such as blood and/or nutrient fluids, from and/or into a patient's body and internal systems.

Referring further to FIGS. 1-6, in one embodiment, a fluid flow control system 10 for transferring fluid, such as blood between an external fluid device or source 12 and a patient's systemic circulatory system (not shown), includes an arteriovenous shunt 14, as shown in FIGS. 2 and 4, that defines a blood flow passage 16. Shunt 14 provides flow communication between an artery and a vein of the patient's systemic circulatory system (not shown). As is known in the art, an arteriovenous shunt facilitates the passage of blood directly from an artery or arteries to a vein or veins, without flowing through the connected capillary network.

In one embodiment, fluid flow control system 10 includes a fluid flow control device 20, such as shown in FIGS. 1-6. Fluid flow control device 20 includes a structural body 22 that forms a channel 24 at least partially extending along a length of structural body 22. In a particular embodiment, channel 24 is positioned about a longitudinal axis 25 of structural body 22, as shown in FIGS. 2 and 4.

Referring to FIGS. 1-6, a sheath 28 is coupled to structural body 22 and positioned about longitudinal axis 25 (FIGS. 2 and 4). Sheath 28 defines a passage 30 in fluid communication with channel 24 that extends between a tip 32 and an opposing base 34 of sheath 28. In one embodiment, sheath 28 is in selective flow communication with channel 24, as described in detail below. Tip 32 is suitably formed for penetrating a tube wall, such as a wall of shunt 14, for introduction into passage 16, as shown in FIGS. 2 and 4. In an alternative embodiment, tip 32 penetrates a patient's skin for subcutaneous introduction of sheath 28 into the patient's body. Sheath 28 is fabricated from at least one suitable biocompatible material including, without limitation, a suitable non-metal, plastic, polymeric, rubber and/or composite material. Such materials facilitate the biocompatibility of sheath 28 with the surrounding body tissues, and prevent or limit undesirable contact with and/or trauma to the surrounding body tissues. Further, suitable materials are resilient and/or pliable while providing the necessary structural integrity for the anticipated applications. In an alternative embodiment, sheath 28 is formed of a suitable metal material, such as a stainless steel or titanium material, which is coated with a suitable biocompatible material. Further, in one embodiment, sheath 28 is fabricated from a suitable material that sealingly couples sheath 28 to shunt 14 to prevent or limit fluid leakage through the coupled members. As shown in FIGS. 2 and 4, fluid flow control device 20 includes a port 36 defining a second passage 38 at a second end 39 of structural body 22, generally opposing sheath 28. Second passage 38 is in flow communication with channel 24.

As shown in FIGS. 1-6, fluid flow control device 20 includes a needle 40 initially positioned within channel 24. In one embodiment, needle 40 is positioned about longitudinal axis 25 and coaxially with respect to sheath 28. Needle 40 is made of any suitable material known to those skilled in the art and guided by the teachings herein provided including, without limitation, the materials described above in reference to sheath 28. Needle 40 is slidably movable at least partially within channel 24 between a first or retracted position and a second or extended position. At least a tip 42 of needle 40 extends outwardly with respect to tip 32 of sheath 28 surrounding a portion of needle 40 to externally position needle 40 with respect to sheath 28. In the extended position, as shown in FIG. 2, at least a portion of needle 40, and at least a portion of sheath 28 surrounding needle 40, can penetrate the patient's skin and/or shunt 14. In a particular embodiment, needle 40 is hollow and defines an inner passage 44 that provides a fluid flow path through at least a portion of fluid flow control device 20. In an alternative embodiment, needle 40 prevents or limits fluid communication between passage 30 formed in sheath 28 and channel 24 in the extended position. For example, needle 40 is fabricated as a solid needle and is configured to prevent fluid flow through needle 40. In the retracted position, as shown in FIG. 2, needle 40 is seated within channel 24 to provide fluid communication between passage 30 and channel 24. In one embodiment, needle 40 is seated within channel 24 along longitudinal axis 25 to allow blood to flow through channel 24 without needle 40 disrupting or interfering with the direction of blood flow. In an alternative embodiment, needle 40 is seated within channel 24 at a distance from longitudinal axis 25 and blood flows around needle 40 and through channel 24.

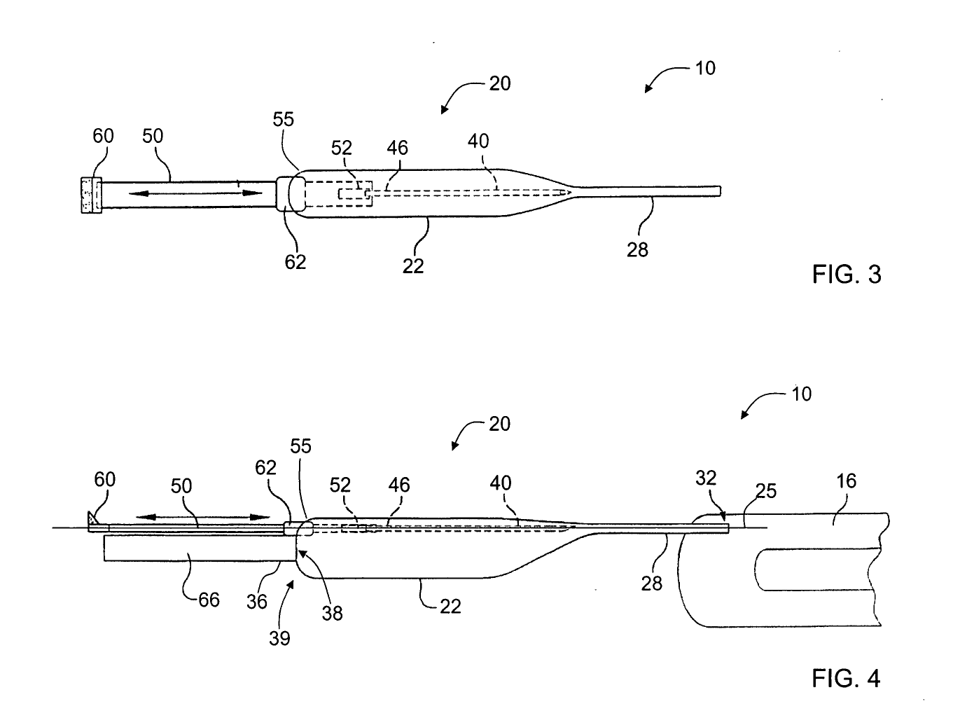

A plate 50 is coupled to needle 40 and is movable with respect to structural body 22 to move needle 40 between the retracted position and the extended position. Plate 50 is made of a suitable material, such as a suitable non-metal, plastic, polymeric, rubber, composite and/or metal material. As shown in FIGS. 1 and 3, in one embodiment, a void 52 is defined between plate 50 and a proximal end 46 of needle 40. In this embodiment, void 52 is in flow communication with inner passage 44 defined by needle 40 to allow blood flashback. Referring further to FIGS. 1-4, in one embodiment, a groove 54 is defined within an end wall 55 of structural body 22. Plate 50 is coupled to proximal end 46 of needle 40 and slidably positioned within groove 54. Plate 50 is movable between a first position to position needle 40 in the retracted position and a second position to position needle 40 in the extended position.

As shown in FIGS. 1-6, a lever 60 is externally positioned with respect to structural body 22 and operatively coupled to needle 40 to move needle 40 between the retracted position seated within channel 24 and the extended position outwardly extended with respect to tip 32 of sheath 28. In a particular embodiment, at least one projection 62, such as at least one ridge, knob and/or raised edge, is positioned on plate 50 and interferes with groove 54 to seal channel 24 to prevent blood from undesirably flowing out of fluid flow control device 20 through groove 54. For example, raised edges on plate 50 partially pass through and engage groove 54 to seal channel 24 to prevent blood leakage from within channel 24 and/or entry of air into channel 24. In an alternative embodiment, a gasket (not shown) or other suitable sealing mechanism seals groove 54 with respect to channel 24 to prevent undesirable fluid flow through groove 54.

In one embodiment, fluid flow control device 20 is operatively coupled to external fluid device 12, such as an external blood source. A fluid transfer tube 66 couples external fluid device 12 with structural body 22. For example, fluid transfer tube 66 is coupled to fluid flow control device 20 at port 36 to provide fluid communication between a passage 68 defined in fluid transfer tube 66 and channel 24 to facilitate transfer of fluids, such as blood, between external fluid device 12 and the patient's body.

In one embodiment, fluid flow control system 10 is configured to transfer blood between an external blood source and a patient's systemic circulatory system (not shown). Arteriovenous shunt 14 defining blood flow passage 16 provides flow communication between an artery and a vein of the systemic circulatory system. In this embodiment, fluid flow control device 20 includes structural body 22 defining channel 24. Sheath 28 is coupled to structural body 22 and defines passage 30 in flow communication with channel 24. In a particular embodiment, plate 50 is slidably positioned within groove 54 formed in structural body 22 and coupled to needle 40.

Needle 40 is movable between the retracted position seated within channel 24 and the extended position outwardly extending with respect to tip 32 of sheath 28. Needle 40 and sheath 28 are at least partially positioned within blood flow passage 16 with needle 40 in the extended position. In one embodiment, needle 40 prevents or limits flow communication between passage 30 defined in sheath 28 and channel 24 in the extended position. Needle 40 is then retracted to remove needle 40 from within blood flow passage 16 to limit undesirable contact between needle 40 and the patient's blood stream and/or the surrounding body tissues. In the retracted position, needle 40 allows or provides flow communication between passage 30 and channel 24. Sheath 28 remains at least partially positioned within blood flow passage 16 when needle 40 is moved to the retracted position to provide continued fluid communication through sheath 28 between shunt 14 and channel 24. Fluid transfer tube 66 provides flow communication between channel 24 and the external blood source.

In one embodiment, lever 60 is externally positioned with respect to structural body 22 and operatively coupled to needle 40 to move needle 40 between the retracted position and the extended position, as desired. In this embodiment, lever 60 is positioned within groove 54 and operatively coupled to needle 40. Lever 60 is movable between the first position wherein needle 40 is in the retracted position and the second position wherein needle 40 is in the extended position.

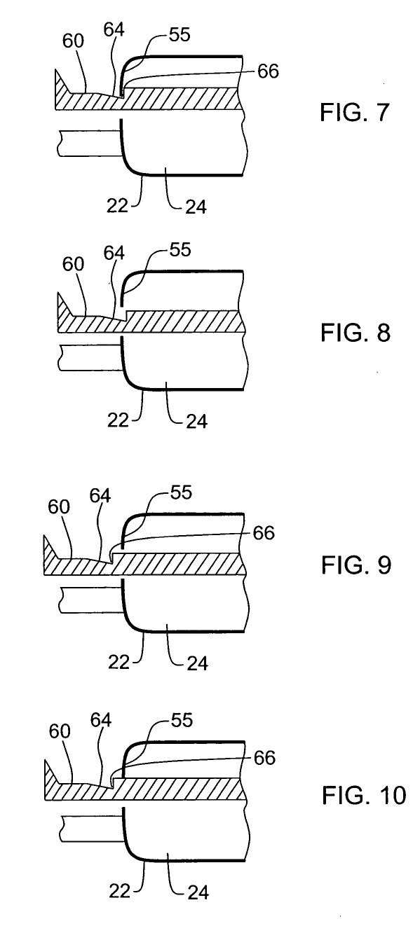

Referring further to FIGS. 7-10, in one embodiment, lever 60 is configured to prevent backward displacement of needle 40 (not shown in FIGS. 7-10) coupled to plate 50, when needle 40 and sheath 28 are inserted into a blood vessel. In this embodiment, lever 60 includes a depression 64, as shown in FIGS. 7-10, that is biased towards end wall 55 of structural body 22. Depression 64 includes a wall 66 that engages or interferes with end wall 55 and, thus, is prevented from backward displacement upon insertion of needle 40 and sheath 28 into the blood vessel.

After successful placement of needle 40 and sheath 28, lever 60 is pushed downwardly towards longitudinal axis 25 of structural body 22 to disengage wall 66 from end wall 55, as shown in FIG. 8. With lever 60 disengaged from structural body 22, lever 60 and needle 40 are withdrawn from within channel 24, as shown in FIGS. 9 and 10. By pushing downwardly on lever 60, wall 66 is disengaged from end wall 55 and plate 50 is freely movable with respect to structural body 22 to withdraw plate 50 without abutting against end wall 55. In a particular embodiment, plate 50 is biased to contact end wall 5 after wall 66 has been disengaged from end wall 55, as shown in FIG. 10. As shown in FIGS. 7-10, groove 54 defined in end wall 55 is configured to allow the downward displacement of lever 60 and easy movement of plate 50 within groove 54. Further, groove 54 is sufficiently large to allow plate 50 to engage structural body 22 at groove 54 and seal channel 24. In this embodiment, needle 40 is prevented from slipping backwards during insertion of needle 40 and sheath 28 into the blood vessel. Further, needle 40 is safely withdrawn following successful insertion of needle 40 and sheath 28 into the blood vessel.

Fluid flow control system 10 and, particularly, fluid flow control device 20, facilitates access to veins, arteries or blood vessels without the need to disconnect the hub, thereby decreasing the risk of blood loss. As a result, any suitable number of sheaths can be placed in veins, arteries and/or blood vessels for or during different procedures. Further, healthcare personnel do not handle exposed needles directly and fluid flow control system 10 can be disposed of safely after the procedure without risk of needlestick injury.

In one embodiment, the present invention provides a method for transferring a fluid between an external fluid source and a patient's body. The method includes inserting needle 40 and sheath 28 surrounding needle 40 into shunt 14. Needle 40 is outwardly extended with respect to tip 32 of sheath 28, with sheath 28 surrounding a portion of needle 40. Sheath 28 defines passage 30 that is in flow communication with channel 24. Lever 60 is operatively coupled to needle 40 and slides from a first position to a second position to extend needle 40 outwardly with respect to tip 32. Needle 40 and sheath 28 penetrate shunt 14 to introduce needle 40 and sheath 28 into fluid flow passage 16 defined by shunt 14. Needle 40 is then retracted to remove needle 40 from within shunt 14. For example, once blood flashes into channel 24, lever 60 is slidably moved from the second position to the first position to retract needle 40. A portion of sheath 28 remains positioned within shunt 14 to provide continued flow communication between passage 30 and channel 24. In a particular embodiment, sheath 28 is fabricated of a suitable material to facilitate sealingly coupling sheath 28 to shunt 14 to prevent or limit undesirable fluid leakage from an opening formed by penetrating the shunt wall, e.g. between an outer surface of sheath 28 and a portion of shunt 14 forming the opening.

The above-described method and apparatus for controlling fluid flow facilitates a continued flow communication between an external fluid source and the patient's internal circulatory system without the invasiveness of a conventional syringe apparatus. More specifically, the method and apparatus facilitate continued flow communication through the fluid flow control device and system by retracting the needle from within the patient's body or a shunt while retaining the sheath, initially surrounding the needle, within the patient's body or the shunt to provide continued flow communication between the external fluid source and the patient's circulatory system. As a result, trauma to the patient's skin, surrounding internal tissues and/or veins is minimized during repeated and/or extended surgical and clinical procedures, such as dialysis.

Exemplary embodiments of a method and apparatus for controlling fluid flow are described above in detail. The method and apparatus is not limited to the specific embodiments described herein, but rather, steps of the method and/or components of the apparatus may be utilized independently and separately from other steps and/or components described herein. Further, the described method steps and/or apparatus components can also be defined in, or used in combination with, other methods and/or apparatus, and are not limited to practice with only the method and apparatus as described herein.

While the invention has been described in terms of various specific embodiments, those skilled in the art will recognize that the invention can be practiced with modification within the spirit and scope of the claims.

What is claimed is:

1. A fluid flow control device comprising:
a structural body forming a channel;
a sheath coupled to a first end of said structural body and having a tip, said sheath defining a passage in flow communication with said channel;
a needle movably positioned within said channel, said needle movable between a retracted position seated within said channel and an extended position extending through said passage and outwardly with respect to said tip; and
a port defining a second passage at an opposing second end of said structural body, said second passage in flow communication with said channel.

2. A fluid flow control device in accordance with claim 1 further comprising a lever operatively coupled to said needle and movable with respect to said structural body to move said needle between the retracted position and the extended position.

3. A fluid flow control device in accordance with claim 1 wherein said device prevents flow communication between said passage and said channel when said needle is in the extended position.

4. A fluid flow control device in accordance with claim 1 wherein said device provides flow communication between said passage and said channel when said needle is in the retracted position.

5. A fluid flow control device in accordance with claim 1 wherein at least a tip of said needle is positioned externally with respect to said sheath in the extended position.

6. A fluid flow control device in accordance with claim 1 further comprising a fluid transfer tube coupled to said port, said fluid transfer tube defining a third passage in flow communication with said second passage.

7. A fluid flow control device in accordance with claim 1 further comprising:
a groove defined within an end wall of said structural body; and
a plate coupled to said needle and at least partially slidably positioned within said groove, said plate movable between a first position wherein said needle is in the retracted position and a second position wherein said needle is in the extended position.

8. A fluid flow control device in accordance with claim 7 further comprising a lever externally positioned with respect to said structural body and coupled to said plate to move said needle between the retracted position seated within said channel and the extended position.

9. A fluid flow control device in accordance with claim 8 wherein said lever defines a depression forming a wall, said wall configured to interfere with said end wall and prevent backward displacement of said plate with respect to said second end.

10. A fluid flow control device in accordance with claim 7 further comprising at least one projection positioned on said plate and interfering with said groove to prevent fluid flow through said groove when said needle is in the retracted position.

11. A fluid flow control system for transferring blood between an external blood source and a patient's systemic circulatory system, said fluid flow control system comprising:
an arteriovenous shunt defining a blood flow passage and providing flow communication between an artery and a vein of said systemic circulatory system;
a fluid flow control device comprising:
a structural body forming a channel;
a sheath coupled to said structural body and defining a passage in flow communication with said channel; and
a needle movably positioned within said channel, said needle movable between a retracted position seated within said channel and an extended position extending beyond a tip of said sheath, each of said needle and said sheath at least partially positioned within said blood flow passage when said needle is in the extended position, said sheath at least partially positioned within said blood flow passage when said needle is in the retracted position; and
a fluid transfer tube providing flow communication between said external blood source and said fluid flow control device.

12. A fluid flow control system in accordance with Claim 11 further comprising a lever externally positioned with respect to said structural body and operatively coupled to said needle to move said needle between the retracted position and the extended position.

13. A fluid flow control system in accordance with claim 11 further comprising:
a groove defined within an end wall of said structural body;
a plate coupled to said needle and at least partially slidably positioned within said groove, said plate movable between a first position wherein said needle is in the retracted position and a second position wherein said needle is in the extended position; and
a void defined between said plate and a proximal end of said needle, said void in flow communication with an inner passage defined by said needle.

14. A fluid flow control system in accordance with Claim 11 wherein said sheath comprises at least one of a non-metal, plastic, polymeric and rubber material.

15. A method for transferring a fluid between an external fluid source and a patient's body, said method comprising:
inserting a needle and a sheath surrounding the needle into a shunt, the sheath coupled to a fluid flow device structural body defining a channel;
retracting the needle to remove the needle from within the shunt;
providing continued flow communication through the sheath between a fluid flow passage formed by the shunt and the channel.

16. A method in accordance with claim 15 further comprising:
moving the needle through a passage formed in the sheath to extend the needle with respect to a tip of the sheath with the sheath surrounding a portion of the needle; and
penetrating the shunt to insert the needle and the sheath into the fluid flow passage.

17. A method in accordance with claim 16 further comprising slidably moving a lever operatively coupled to the needle from a first position to a second position to extend the needle from an end of the sheath.

18. A method in accordance with claim 17 wherein retracting the needle to remove the needle from within the shunt further comprises slidably moving the lever from the second position to the first position.

19. A method in accordance with claim 15 further comprising sealingly coupling the sheath to the shunt to substantially prevent fluid leakage.

20. A method in accordance with claim 15 further comprising controlling a flow communication through the fluid flow control device by moving the needle between the first position allowing flow communication between a passage defined by the sheath and the channel, and the second position preventing flow communication between the passage and the channel.
